(19) United States
(12) Patent Application Publication
(10) Pub. No.: US 2023/0277338 A1
(43) Pub. Date: Sep. 7, 2023

(54) WATERTIGHT FINGER ELEMENT

(71) Applicant: Stefan SCHULZ, Karlsruhe (DE)

(72) Inventor: Stefan SCHULZ, Karlsruhe (DE)

(21) Appl. No.: 18/000,585

(22) PCT Filed: Jun. 6, 2021
(86) PCT No.: PCT/DE2021/100486

§ 371 Date: Mar. 14, 2023

(30) Foreign Application Priority Data
Jun. 5, 2020 (DE) 10 2020 115 099.7

(51) Int. Cl. A61F 2/58

(52) U.S. Cl. CPC A61F 2/586; A61F 2002/587; A61F 2002/5015

(57) ABSTRACT

Finger element or thumb element with at least a sleeve which defines a volume and in which at least one motor is arranged, such that the said volume is at least partially filled with a filler material.

### FIELD OF THE INVENTION

The invention relates to a finger element and/or a thumb element for a hand prosthesis and to a hand prosthesis comprising a finger element and/or a thumb element.

### PRIOR ART

Hand prostheses are used to replicate natural hands or the function of natural hands. Thus for example, hand prostheses have a number of fingers which can comprise one or more finger phalanges. These phalanges can for example be moved by one or more electric motors. The motor can be positioned in the phalanx, particularly in the first phalanx. Furthermore the hand prosthesis can comprise a thumb element that has a number of thumb phalanges. The finger and thumb phalanges can be connected to one another by way of a palm element. By way of the palm element the finger and thumb phalanges can also be connected to a wrist element. Hand prostheses can be adapted to suit the corresponding user. Thus, hand prostheses of various sizes and shaped can be produced. Furthermore, in modern hand prostheses the left and the right hand prosthesis differ in their shape. Thus for example, the left hand prosthesis is often made as a mirror-image of the right hand prosthesis.

### OBJECTIVE

The purpose of the present invention is to make possible a new and inventive finger or thumb phalanx. A further purpose of the invention can be to design the hand prosthesis more robustly.

### DESCRIPTION OF THE INVENTION

The stated objective is achieved by a finger phalanx and/or a thumb phalanx according to the first independent claim and by a hand prosthesis according to the further independent claim.

In particular, the objective can be achieved by a finger and/or thumb phalanx with at least one sleeve which defines a volume and within which at least one motor is arranged, the volume being at least partially filler with a filler material.

Preferably the filler is a gel. The filler can in that case be introduced easily into the sleeve and can have a viscosity such that it remains in the sleeve.

Preferably, after being introduced into the sleeve the gel can change its viscosity, in particular becoming solid or more viscous.

Preferably, the filler is not soluble in water. The filler can then be suitable for making the phalanx watertight; in particular the motor and/or any gearing present in the volume are protected against water.

### BRIEF DESCRIPTION OF THE DRAWINGS

In what follows the invention will be described in greater detail with reference to example embodiments, with the aid of drawings, which show:

FIG. 1: A hand prosthesis,

FIG. 2: A hand prosthesis without its sleeve, viewed laterally from above,

FIG. 3: A hand prosthesis without its sleeve, viewed from above,

FIG. 4: A hand prosthesis without its sleeve, viewed from the left,

FIG. 5: A hand prosthesis without its sleeve, viewed from below,

FIG. 6: A hand prosthesis without its sleeve, viewed from the right,

FIG. 7: Exploded view of a hand prosthesis,

FIG. 8: The hand prosthesis shown in FIG. 7, as an exploded view seen from below,

FIG. 9: An exploded view of a hand prosthesis with various finger frame elements,

FIG. 10: A metacarpus element

FIG. 11: Exploded view of the metacarpus element shown in FIG. 10,

FIG. 12: A metacarpus element viewed from the front,

FIG. 13: An open metacarpus element viewed from the front,

FIG. 14: An exploded view of a hand prosthesis with movement dampers,

FIG. 15: The hand prosthesis of FIG. 14, viewed from one side,

FIG. 16: The hand prosthesis of FIG. 14, viewed from below,

FIG. 17: A sectioned view of a finger element.

### DETAILED DESCRIPTION OF THE DRAWINGS

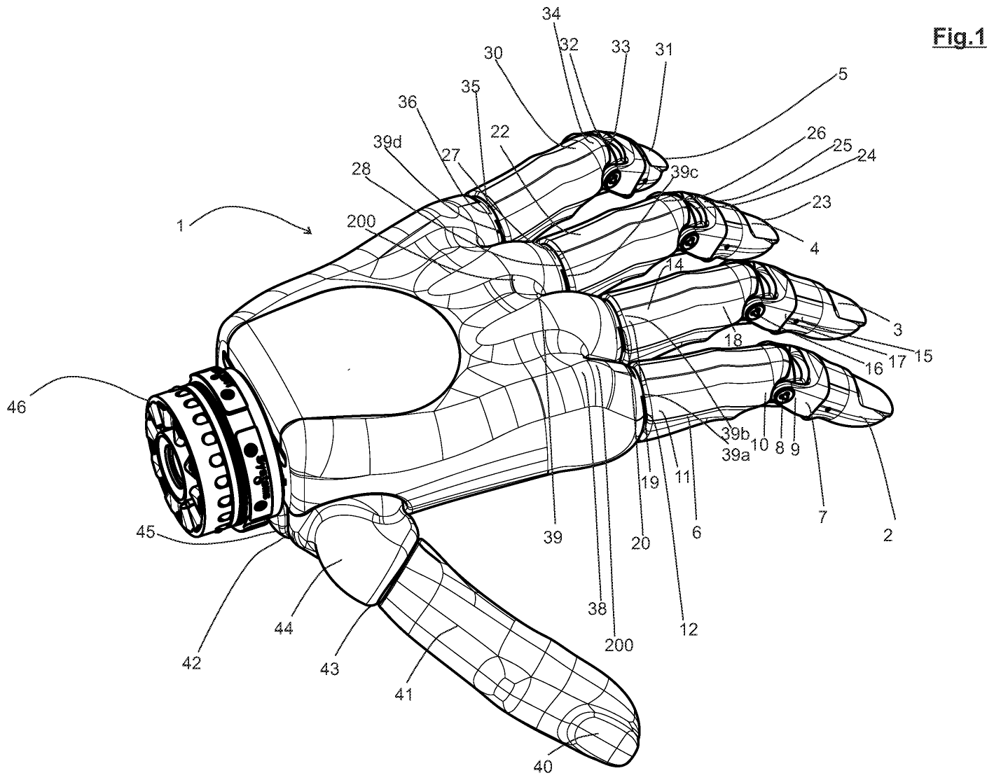

FIG. 1 shows a hand prosthesis 1 with a first finger element 2, a second finger element 3, a third finger element 4 and a fourth finger element 5. The first finger element 2 has a first, proximal phalanx 6 and a first, distal phalanx 7. The first distal phalanx 7 is connected to a distal end 10 of the first proximal phalanx 6 by means of a finger joint 8 at a proximal end 9 of the first distal phalanx 7, about which joint it can pivot. A proximal end 11 of the first proximal phalanx 6 is connected by means of a first proximal finger joint 12 to a first phalanx base member 13 (not shown here).

The second finger element 3 has a second proximal phalanx 14 and a second distal phalanx 15. The second distal phalanx 15 is connected pivotably by way of a second distal finger joint 16 at a proximal end 17 of the second distal phalanx 15 to a distal end 18 of the second proximal phalanx 14. A proximal end 19 of the second proximal phalanx 14 is connected by means of a second proximal finger joint 20 to a second phalanx base member 21 (not shown here).

The third finger element 4 has a third proximal phalanx 22 and a third distal phalanx 23. The third distal phalanx 23 is connected pivotably by way of a third distal finger joint 24 at a proximal end 25 of the third distal phalanx 23 to a distal end 26 of the third proximal phalanx 22. A proximal end 27 of the third proximal phalanx 23 is connected by means of a third proximal finger joint 28 to a third phalanx base member 29 (not shown here).

The fourth finger element 5 has a fourth proximal phalanx 30 and a fourth distal phalanx 31. The fourth distal phalanx 31 is connected pivotably by way of a fourth distal finger joint 32 at a proximal end 33 of the fourth distal phalanx 31 to a distal end 34 of the fourth proximal phalanx 31. A proximal end 35 of the fourth proximal phalanx 30 is connected by means of a fourth proximal finger joint 36 to a fourth phalanx base member 37 (not shown here).

Furthermore, the hand prosthesis 1 has a sleeve 38 made of a soft plastic material such as silicone. The sleeve 38 at least partially covers the proximal finger joints 12, 20, 28 and 36, for example in order to prevent any object from making its way into the proximal finger joints 12, 20, 28 and 36 or between the metacarpal element 200 and any of the proximal phalanges 6, 14, 22, 30.

In its distal area 39 the sleeve 38 has four openings 39a, b, c, d. The first finger element 2 projects through the first opening 39a, the second finger element 3 through the second opening 39b, the third finger element 4 through the third opening 39c and the fourth finger element 5 through the fourth opening 39d. The openings 39a, 39b, 39c, 39d can be arranged approximately in a row in accordance with the anatomical positions of the phalanx base joints. The openings 39a, b, c, d can thus hold the corresponding finger elements 2, 3, 4, 5 in their proximal area and in that way, for example, damp or restrict a movement of the proximal area relative to the metacarpal element 200. In particular, a pivoting movement of one or more of the finger elements 2, 3, 4, 5 in a main plane of the metacarpal element 200 can be damped by the sleeve 38.

In addition the hand prosthesis 1 has a thumb element 40 with a distal thumb phalanx 41 and a proximal thumb phalanx 42. The thumb element 40 can also extend through an opening in the sleeve 38, or the sleeve 38 is clear of the thumb element so that the sleeve 38 can be pulled off the metacarpal element 200.

The distal thumb phalanx 41 is connected to the proximal thumb phalanx 42 by way of a distal thumb joint 43. In this case the distal thumb joint 43 is covered by a cap 44. The cap 44 is approximately tubular with two opposite openings, such that the distal thumb phalanx 41 extends through one opening and the proximal thumb phalanx 42 extends through the second opening. The cap 44 is thus held by the two thumb phalanges 41 and 42 and the thumb joint 43.

At the proximal end 45 of the hand prosthesis 1 there is a wrist element 46, which can be connected to an arm stump.

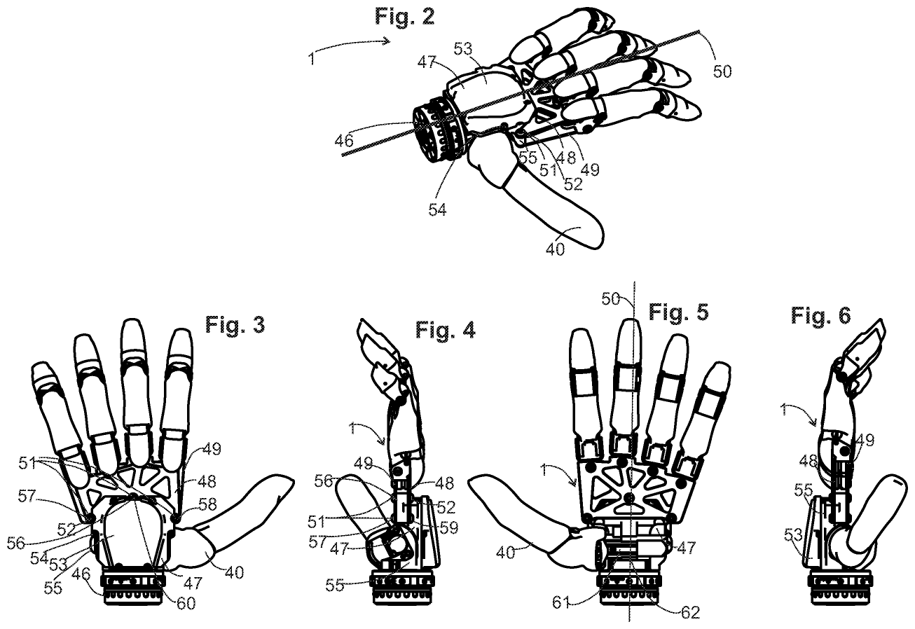

FIG. 2 shows the hand prosthesis 1 without its sleeve 38, viewed obliquely from above. The hand prosthesis 1 in this case comprises a metacarpal element 47, which is connected to the wrist element 46, a first finger frame element 48, a second finger frame element 49 and the thumb element 40. The metacarpal element 47 is mirror-symmetrical relative to its main axis 50. The main axis 50 of the metacarpal element 47 extends from the proximal toward the distal.

The metacarpal element 47 has a connection element 51 for connecting the metacarpal element 47 to the finger frame elements 48 and 49. In this case the connection element 51 has a bore through which a screw 52 is passed.

The metacarpal element 47 comprises a metacarpal cover 53, which for example is connected to a metacarpal base element 55 by the screw connection 54.

The metacarpal base element 55 and/or the metacarpal cover 53 can be made at least in part from a rigid material such as metal, plastic and/or a composite. In particular the metacarpal base element 55 and/or the metacarpal cover 53 can be transparent, at least in part.

FIG. 3 shows the hand prosthesis 1 without its sleeve, viewed from above. FIG. 4 shows the hand prosthesis 1 viewed from the right side. In this it can be seen that the finger frame element 48 and the finger frame element 49 are connected to the metacarpal element 47, in particular the metacarpal base element 55, by the connection element 51. The connection element 51 comprises the connection part-elements 56, 57, 58, which in this case are each in the form of bores. A respective screw 52, 59, 60 is passed through the connection part-elements 56, 57 and 58.

FIG. 5 shows the hand prosthesis 1 viewed from below. It can be seen that a thumb holding member 61 is held in a thumb holder 62. The thumb holder 62 is mirror-symmetrical relative to the main axis 50, so that the thumb phalanx 40 can be attached to the metacarpal element 47 both from the left and from the right side.

FIG. 6 shows the hand prosthesis 1 viewed from the left side. In the figure can be seen the metacarpal cover 3, which is attached to the metacarpal base element 55. It can also be seen that the finger frame elements 48, 49 extend in parallel planes, in particular parallel to the main plane of the metacarpal element 200, and are attached to the metacarpal base element 55.

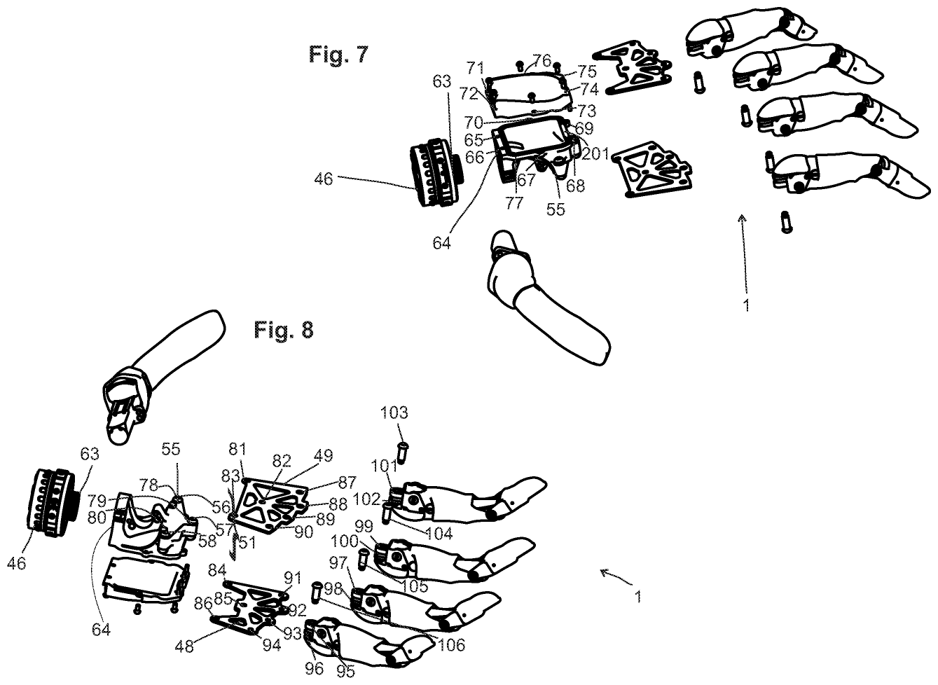

FIGS. 7 and 8 shows exploded views of the hand prosthesis 1. The hand prosthesis 1 comprises the wrist base element 46, which can be attached to the metacarpal base element 55 by means of a cylindrical projection 63. The projection 63 is inserted into an opening 64 which has a diameter similar to or if necessary slightly larger than the projection 63. By virtue of bores running from the outside through the metacarpal base element 55 to the opening 64 screws can extend, which hold the projection 63 in the opening 64 and thereby connect the wrist element 46 to the metacarpal base element 55.

The metacarpal base element 55 has a number of threaded bores 65, 66, 67, 68, 69, 70 whose position corresponds to the bores 71, 72, 73, 74, 75, 76 in the metacarpal cover 53. Through the bores 71, 72, 73, 74, 75, 76 screws can be introduced into the threaded bores 65, 66, 67, 68, 69, 70 and in that way connect the metacarpal base element 55 to the metacarpal cover 53. In this case a seal 77 is arranged between the metacarpal base element 55 and the metacarpal cover 53, which seals the gap between the metacarpal base element 55 and the metacarpal cover 53.

In its distal area the seal 77 has a break 201 through which cables can pass. These cables can serve to pass electrical signals to one or more motors in order to move one or more of the finger elements. The cables can also supply current to the motor or motors.

In addition the metacarpal base element 55 comprises the connection element 51 with its connecting part-elements 56, 57, 58. These connecting part-elements 56, 57, 58 are bores through which the screws 78, 79, 80 are passed. The arrangement of the connecting part-elements 56, 57, 58 corresponds to the arrangement of the bores 81, 82, 83 in the first finger frame element 48 and the arrangement of the bores 84, 85, 86 in the second finger frame element 49. The screws 78, 79, 80 can for example have a thread and be held in a thread of the bores 84, 85, 86 or 81, 82, 83, or be connected by means of a nut on the opposite side, so that the first finger frame element 48, the metacarpal base element 55 and the second finger frame element 49 are held between the screw head and the nut. In this case the metacarpal base element 55 is arranged between the first finger frame element 48 and the second finger frame element 49.

In their distal area the finger frame elements 48, 49 have four finger element holding bores in each case, namely 87, 88, 89, 90 and 91, 92, 93, 94 respectively. The finger element holding bores 87, 88, 89, 90 of the first finger frame element 48 are equally spaced from one another, as are the finger element holding bores 91, 92, 93, 94.

At the proximal end of the finger elements 2, 3, 4, 5 there are in each case a first tongue 95, 97, 99, 101 and a second tongue 96, 98, 100, 102. The tongues have in each case an opening though which a respective screw 103, 104, 105, 106 can be passed. The screws 103, 104, 105, 106 have cylindrical screw shanks provided in part or all over with a thread and a screw head arranged at the end of the screw shank. Furthermore, the screws 103, 104, 105, 106 each have a nut that can be screwed onto the screw shank, or alternatively the thread can be in the bores 87, 88, 89, 90 of the finger frame element 48 or the bores 91, 92, 93, 94 of the finger frame element 49.

In the condition when it is in use the first finger frame element 48 is on the upper side of the second tongues 96, 98, 100, 102 and the second finger frame element 49 is on the lower side of the first tongues 95, 97, 99 and 101.

The first finger frame element 48 is thus arranged between nuts, which can be omitted if the threaded bores 87, 88, 89, 90 are present, and the second tongues 96, 98, 100, 102, and the second finger frame element 49 is between the screw heads and the first tongues 95, 97, 99 and 101.

The finger elements 2, 3, 4, 5 can pivot about an axis which is perpendicular to the plane of the finger elements 48, 49 and corresponds to the main axes of the screws 103, 104, 105, 106. The pivoting movement can be damped by the sleeve 38. Above a certain amplitude of the pivoting movement the finger base member of the respective finger element 2, 3, 4, 5 comes up against one or other of the two finger frame elements 48, 49, so that the pivoting movement of the finger elements 2, 3, 4, 5 is limited. During this the maximum amplitude of the pivoting movement can vary depending on the position of the finger elements 2, 3, 4, 5, i.e. whether for example it is in a position corresponding to the first finger element 2, namely the index finger, or the middle finger. The maximum amplitude of the pivoting movement can also be different in one direction from the maximum amplitude of the pivoting movement can also be different in the other direction. The neutral position of the finger elements is defined by a finger guide or the openings 39a, 39b, 39c, 39d of the sleeve 38.

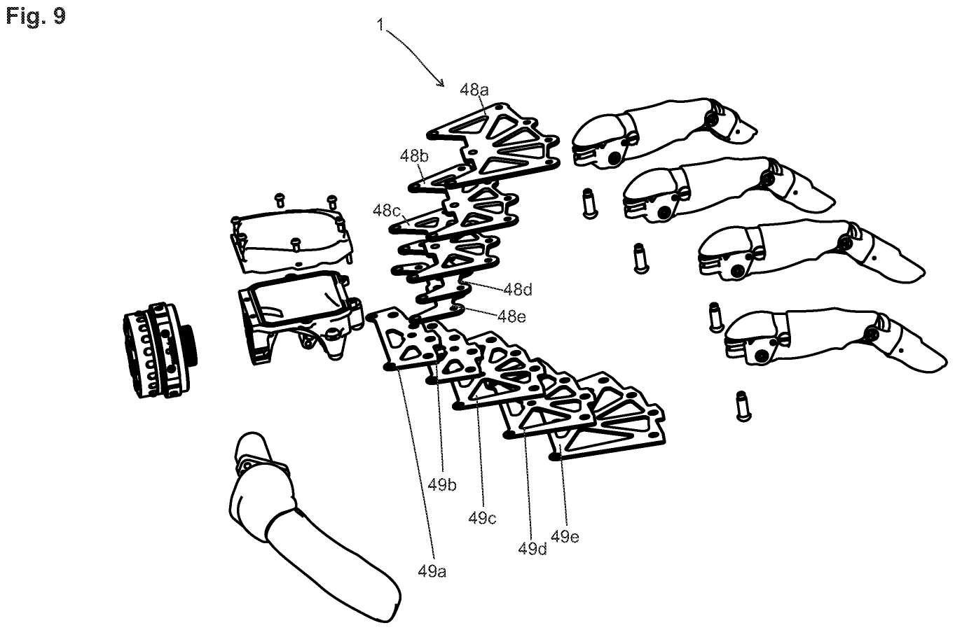

FIG. 9 shows an exploded view of the hand prosthesis 1 with variously sized finger frame elements 48a, 48b, 48c, 48d, 48e, 49a, 49b, 49c, 49d, 49e. It should be noted that by virtue of the different sizes the connection elements for connecting the finger frame elements 48, 49 to the metacarpal base element remain the same, in particular the positions of the bores of the finger frame elements 48, 49 that correspond with the connection part-elements.

The second finger frame elements 49a, b, c, d and e are of various sizes and each comprises the stops for limiting the pivoting movement.

The first finger frame element 48a is used with the second finger frame element 49a, the first finger frame element 48b is used with the second finger frame element 49b, and so on.

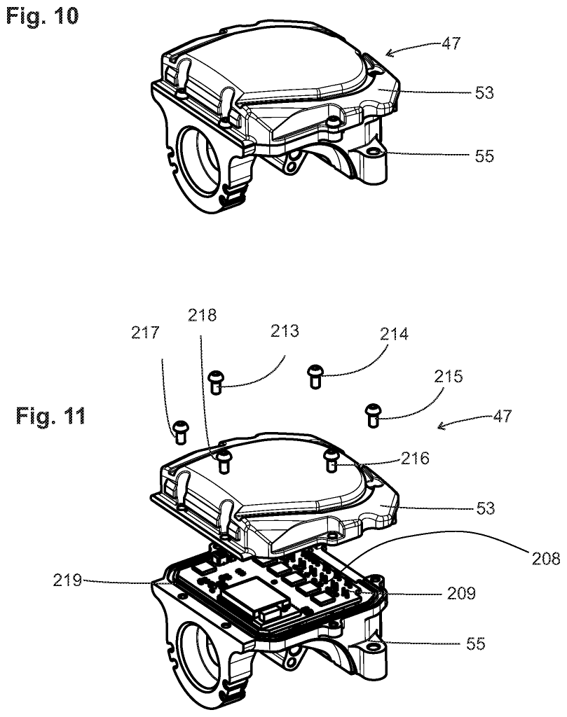

FIG. 10 shows a metacarpal element 47 and FIG. 11 is an exploded view of the metacarpal element 47. FIG. 11 shows the metacarpal cover 53, which is attached to the metacarpal base element 55 by screws 214, 215, 216, 217 and 218. Between the metacarpal cover 53 and the metacarpal base element 55 there is arranged a seal 219, in particular a closed or open sealing ring. Thus, the metacarpal cover 53 together with the metacarpal base element 55 enclose a volume 208, which accommodates a control unit 209. The control unit 209 can therefore be protected against moisture.

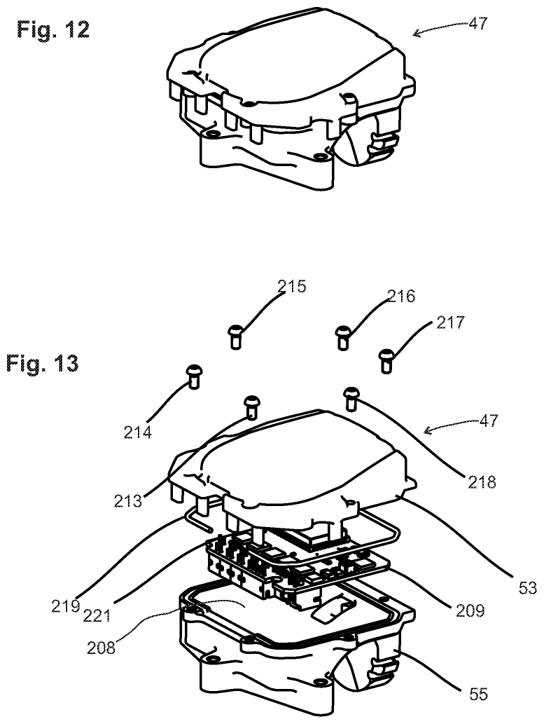

FIG. 12 shows the metacarpal element 47 in a closed condition.

FIG. 13 shows an exploded view of the metacarpal element 47.

In the figures the metacarpal cover 53 can be seen, together with the metacarpal base element 55. Between the metacarpal cover 53 and the metacarpal base element 55 is the seal 219, in particular a sealing ring for example made of silicone. The seal 219 has a gap 221 for cables, through which cables ca be passed outward from the control unit. In the area of the cable passage the seal can be broken and the cables passing through can be enclosed by a water-insoluble gel. Thus, the volume 208 can be resealed.

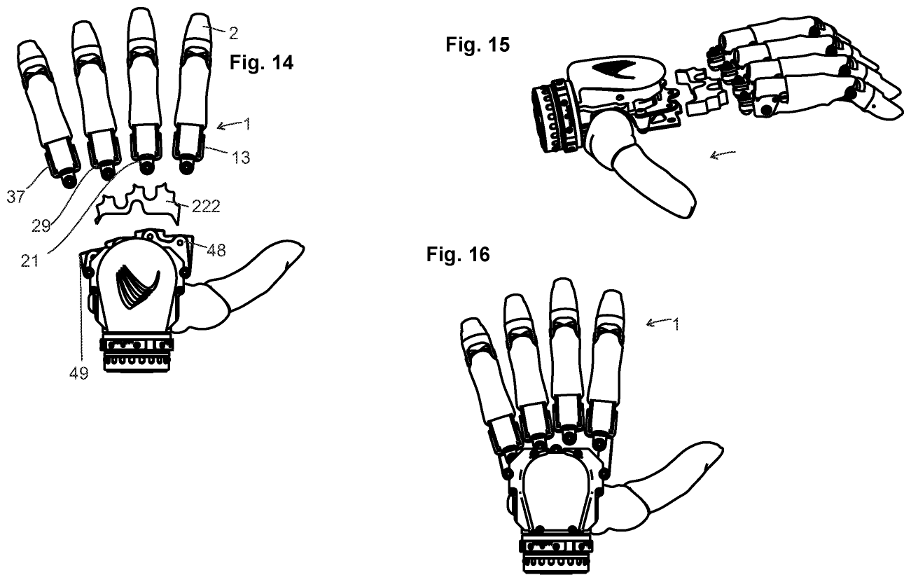

FIG. 14 shows a hand prosthesis 1 with a movement damper 222. In this case the movement damper 222 is held between the finger base members 13, 21, 29, 31 and the finger frame elements 48, 49. The movement damper 222 is made of a flexible material such as rubber or silicone and is in contact with the finger base members 13, 21, 29, 31 so that each finger base member 13, 21, 29, 31 is pushed to a neutral pivoting position and a pivoting movement, for example of the finger element 2 in the main plane of the hand prosthesis 1 (here, the plane of the drawing) is damped.

FIG. 15 shows the hand prosthesis of FIG. 14, viewed from the side.

FIG. 16 shows the hand prosthesis of FIG. 14, viewed from below.

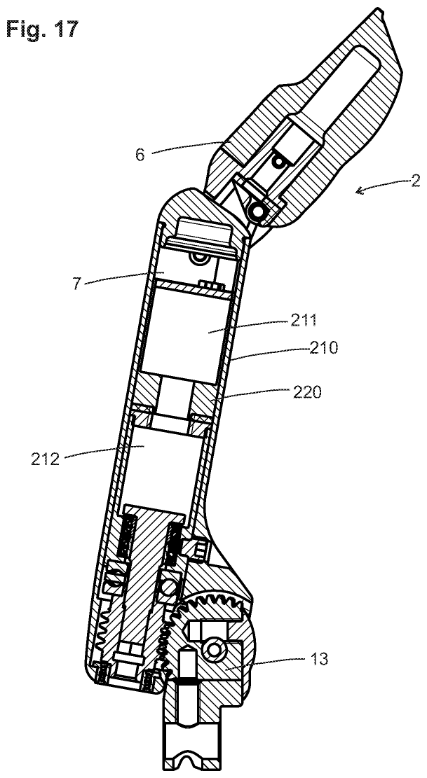

FIG. 17 shows a sectioned view of a finger element 2. In this case the finger element comprises the distal phalanx 6, the proximal phalanx 7 and the base member 13 of the finger. The proximal phalanx 7 has a shell 210 inside which an electric motor 211 with connected gearing 212 are accommodated. The shell also contains a water-insoluble gel 220 which at least partially surrounds the electric motor 211 and the gearing 212 and thereby seals at least the electric motor 211 in a watertight manner.

The gel 220 can be introduced into the shell 210 in liquid form while the electric motor 211 and the gearing 212 are already in the shell. The gel 220 can then set and in that way seal at least some parts of the shell 210 in a watertight manner.

### INDEXES

1 Hand prosthesis
2 First finger element
3 Second finger element
4 Third finger element
5 Fourth finger element
6 First proximal phalanx
7 First distal phalanx
8 First distal finger joint
9 Proximal end of the first distal phalanx
10 Distal end of the first proximal phalanx
11 Proximal end of the first proximal phalanx
12 First proximal finger joint
13 First phalanx base member
14 Second proximal phalanx
15 Second distal phalanx
16 Second distal finger joint
17 Proximal end of the second distal phalanx
18 Distal end of the second proximal phalanx
19 Proximal end of the second proximal phalanx
20 Second proximal finger joint
21 Second phalanx base member
22 Third proximal phalanx
23 Third distal phalanx
24 Third distal finger joint
25 Proximal end of the third distal phalanx
26 Distal end of the third proximal phalanx
27 Proximal end of the third proximal phalanx
28 Third proximal finer joint
29 Third phalanx base member
30 Fourth proximal phalanx
31 Fourth distal phalanx
32 Fourth distal finger joint
33 Proximal end of the fourth distal phalanx
34 Distal end of the fourth proximal phalanx
35 Proximal end of the fourth proximal phalanx
36 Fourth proximal finger joint
37 Fourth phalanx base member
38 Sleeve
39 Distal area of the sleeve
40 Thumb element
41 Distal thumb phalanx
42 Proximal thumb phalanx
43 Distal thumb joint
44 Cap
45 Proximal end of the hand prosthesis
46 Write element
47 Metacarpal element
48 First finger frame element
49 Second finger frame element
50 Main axis of the metacarpal element
51 Connection element
52 Screw
53 Metacarpal cover
54 Screw connection
55 Metacarpal base element
56 Connection part-element
57 Connection part-element
58 Connection part-element
59 Screw
60 Screw
61 Thumb holding member
62 Thumb holder
63 Projection
64 Opening
65 Threaded bores
66 Threaded bores
67 Threaded bores
68 Threaded bores
69 Threaded bores
70 Threaded bores
71 Bore
72 Bore
73 Bore
74 Bore
75 Bore
76 Bore
77 Seal
78 Screw
79 Screw
80 Screw
81 Bore
82 Bore
83 Bore
84 Bore
85 Bore
86 Bore
87 Finger element holding bore
88 Finger element holding bore
89 Finger element holding bore
90 Finger element holding bore
91 Finger element holding bore
92 Finger element holding bore
93 Finger element holding bore
94 Finger element holding bore
95 First tongue
96 Second tongue
97 First tongue
98 Second tongue
99 First tongue
100 Second tongue
101 First tongue
102 Second tongue
103 Screw
104 Screw
105 Screw
106 Screw
200 Palm element
201 Break in the seal
202 Screw
203 Screw
204 Screw
205 Screw
206 Screw
207 Screw
208 Volume
209 Control unit
210 Shell
211 Electric motor
212 Gearing
213 Screw
214 Screw
215 Screw
216 Screw
217 Screw
218 Screw
219 Seal
220 Gel
221 Cable passage

1. A finger element or thumb element with at least a sleeve which defines a volume and in which at least one motor is arranged, wherein the volume is at least partially filled with a filler material.

2. The finger element or thumb element according to claim 1, wherein the motor and/or gearing, in particular motor gearing, is/are surrounded by the filler material.

3. The finger element or thumb element according to claim 2, wherein the filler material is a gel.

4. The finger element or thumb element according to claim 1, wherein the filler material is not soluble in water.

5. The finger element or thumb element according to claim 1, wherein after it has been introduced, the filler material becomes viscous or hard.

6. A hand prosthesis with a finger element or thumb element according to claim 1.
